(19) United States
(12) Patent Application Publication
(10) Pub. No.: US 2008/0115509 A1
(43) Pub. Date: May 22, 2008

(54) Methods and Apparatus for Forming and Connecting Cryoprobes for use with Cryosurgical Treatment Systems

(76) Inventors: Adam L. Gullickson, Richfield, MN (US); Douglas A. Devens, Highland Park, IL (US); David W. Vancelette, San Diego, CA (US); Michael W.V. Perkins, Minnetonka, MN (US); Randall C. Lieser, Plymouth, MN (US)

Correspondence Address: AMS RESEARCH CORPORATION, 10700 BREN ROAD WEST, MINNETONKA, MN, 55343, US

(21) Appl. No.: 11/942,029

(22) Filed: Nov. 19, 2007

Related U.S. Application Data
(60) Provisional application No. 60/866,273, filed on Nov. 17, 2006.

(51) Int. Cl. F17C 13/00; A61B 18/02

(52) U.S. Cl. 62/50.7; 606/20

(57) ABSTRACT

Methods and apparatus for connecting disposable and non-disposable portions of a cryoprobe for use with a cryosurgical treatment system. Representative cryoprobes can include a disposable and non-disposable portion joined with a connector such as a coupler for connecting a single disposable portion to a single non-disposable portion. A representative coupler can include coupler ports into which connecting ends of fluid delivery tubes within the disposable and non-disposable portions can fluidly connect. A manifold mounting plate can include a plurality of slots into which disposable portions can be fluidly interconnected to non-disposable portions.

### PRIORITY CLAIM

The present application claims priority to U.S. Provisional Application Ser. No. 60/866,273, filed Nov. 17, 2006 and entitled “METHODS AND APPARATUS FOR FORMING AND CONNECTING CRYOPROBES FOR USE WITH CRYOSURGICAL TREATMENT SYSTEMS”, which is herein incorporated by reference in its entirety.

### FIELD OF THE INVENTION

The present disclosure relates to cryosurgical systems for treatment of benign and cancerous tissues. In particular, the present disclosure relates to apparatus and methods for connecting a disposable portion of a cryoprobe for use in a cryosurgical system to a non-disposable cryoprobe portion.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer and benign prostate disease. Cryosurgery also has gynecological applications. In addition, cryosurgery may be used for the treatment of a number of other diseases and conditions including, but certainly not limited to, breast cancer, liver cancer, renal cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

### SUMMARY OF THE INVENTION

The present disclosure is directed to methods and apparatus for connecting disposable and non-disposable portions of a cryoprobe for use with a cryosurgical treatment system. In some representative embodiments, the disposable and non-disposable portion can include connecting means such as, for example, a coupler for connecting a single disposable portion to a single non-disposable portion. A representative coupler can include coupler ports into which connecting ends of fluid delivery tubes within the disposable and non-disposable portions can fluidly connect. In other representative embodiments, the connecting means, and more particularly the non-disposable portions can comprise a manifold mounting plate. The manifold mounting plate can include a plurality of slots into which disposable portions can be fluidly interconnected to non-disposable portions.

In one aspect of the present disclosure, a coupler can be used to interconnect individual disposable and non-disposable cryoprobe portions. The coupler can provide a first port into which fluid can flow from a delivery tube in the non-disposable portion into a capillary tube or other Joule-Thompson expansion element in the disposable portion. After the cooling effects of the refrigerant has been utilized at a tip of the disposable cryoprobe portion, the refrigerant can flow from a return channel in the disposable portion into a corresponding return channel in the non-disposable portion through a second coupler port. In some representative embodiments, refrigerant flow through the coupler ports can be coaxial. In other representative embodiments, flow through the coupler ports can be oriented in a side by side configuration.

In another aspect of the present disclosure, a manifold mounting plate can be used to connect a plurality of disposable and non-disposable cryoprobe portions to form a plurality of cryoprobes for use in a cryosurgical treatment. The manifold mounting plate can be disposed on an articulating arm of a cryosurgical system. Each disposable portion can plug into a manifold slot of the manifold mounting plate to connect each disposable portion with a non-disposable portion. Refrigerant can then flow through delivery tubes within the non-disposable portions into capillary tubes within the disposable portions and through return channels within the disposable portions into return channels within the non-disposable portions. In some representative embodiments, vacuum insulation can be built into the manifold mounting plate, with a single line of insulation surrounding all of the non-disposable portions. Alternatively, the manifold mounting plate can be fabricated such that vacuum insulation surrounds each individual non-disposable portion.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a cryosurgical system according to the present disclosure.

FIG. 2 is a schematic, section view of a portion of an embodiment of a cryoprobe according to the present disclosure.

FIG. 3 is a schematic, section view of a portion of an embodiment of a cryoprobe according to the present disclosure.

FIG. 4 is a schematic, section view of a portion of an embodiment of a cryoprobe according to the present disclosure.

FIG. 5 is a schematic, section view of a portion of an embodiment of a cryoprobe according to the present disclosure.

FIG. 6 is a cross sectional view of a connecting portion of a manifold mounting plate according to an embodiment of the present disclosure.

FIG. 7 is a perspective, end view of the manifold mounting plate of FIG. 6.

FIG. 8 is a cross sectional view of a connecting portion of a manifold mounting plate according to an embodiment of the present disclosure.

FIG. 9 is a perspective, end view of an embodiment of the manifold mounting plate of FIG. 8.

### DETAILED DESCRIPTION

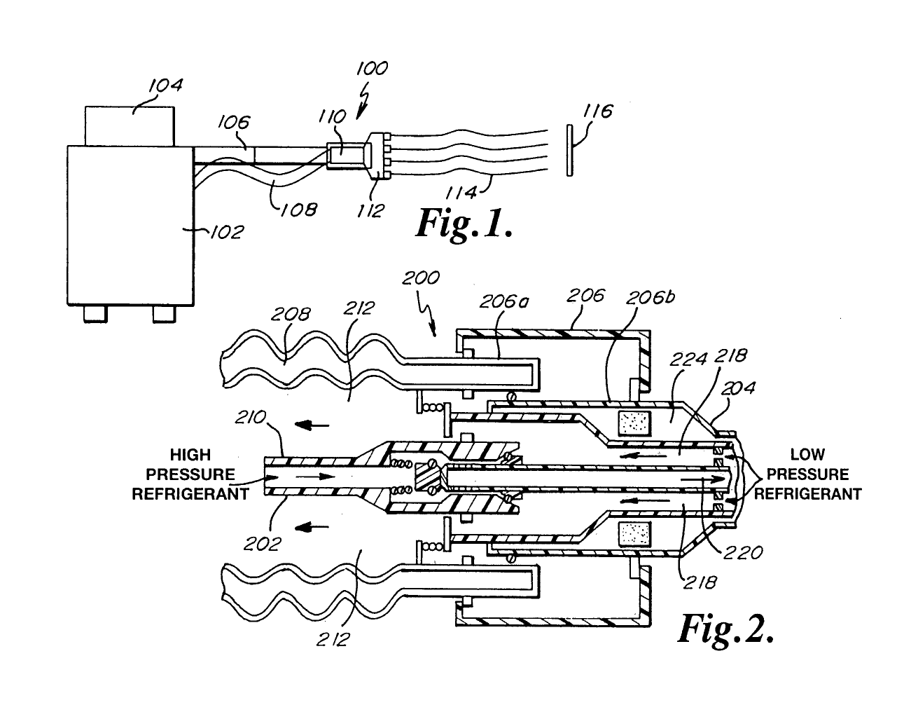

A closed loop cryosurgical system 100 according to the present disclosure is illustrated generally in FIG. 1. Cryosurgical system 100 can include a refrigeration and control console 102 with an attached display 104. Control console 102 can contain a primary compressor to provide a primary pressurized, mixed gas refrigerant to the system and a secondary compressor to provide a secondary pressurized, mixed gas refrigerant to the system. The use of mixed gas refrigerants is generally known in the art to provide a dramatic increase in cooling performance over the use of a single gas refrigerant. Control console 102 can also include controls that allow for the activation, deactivation, and modification of various system parameters, such as, for example, gas flow rates, pressures, and temperatures of the mixed gas refrigerants. Display 104 can provide the operator the ability to monitor, and in some embodiments adjust, the system to ensure it is performing properly and can provide real-time display as well as recording and historical displays of system parameters. One exemplary console that can be used with an embodiment of the present invention is used as part of the Her Option® Office Cryoablation Therapy available from American Medical Systems of Minnetonka, Minn.

With reference to FIG. 1, the refrigerant can be transferred from control console 102 to a cryostat heat exchanger module 110 through a flexible line 108. The cryostat heat exchanger module 110 can include a manifold portion 112 that transfers refrigerant into and receives refrigerant out of a plurality of cryoprobes 114. Alternatively, each cryoprobe 114 can be individually connected to separate refrigerant lines. The cryostat heat exchanger module 110 and cryoprobes 114 can also be connected to the control console 102 by way of an articulating arm 106, which can be manually or automatically used to position the cryostat heat exchanger module 110 and cryoprobes 114. Although depicted as having the flexible line 108 as a separate component from the articulating arm 106, cryosurgical system 100 can incorporate the flexible line 108 within the articulating arm 106. A positioning grid 116 can be used to properly align and position the cryoprobes 114 for patient insertion.

As illustrated in FIG. 2, a representative cryoprobe that can be used with a cryosurgical system according to the present disclosure can comprise a non-disposable base portion 202 and a disposable end portion 204 that can connect to one another with a coupler 206. Disposable portion 204 can be entirely straight and rigid or can have a flexible end. Coupler 206 can be a two part coupler having a first coupler portion 206a integral with non-disposable portion 202 and a second coupler portion 206b integral with disposable portion 204.

As can be seen in FIG. 2, cryoprobe 200 can include first coupler portion 206a and second coupler portion 206b arranged in a coaxial configuration for delivering high pressure refrigerant into disposable portion 204 and for returning the resulting low pressure refrigerant through non-disposable portion 202. High pressure refrigerant can enter a delivery tube 210 in non-disposable portion 202 and flow into a capillary tube 220, or other Joule-Thompson expansion element, that can be connected to delivery tube 210 by coupler 206. The high pressure refrigerant is expanded as it exits the capillary tube 220 and can then be used to form ice balls on a conductive freeze tip of the cryoprobe in order to perform a cryothermal treatment.

Once the expanded, low pressure refrigerant has been used to cool the conductive freeze tip for cryosurgical treatment the low pressure refrigerant returns to control console 102 through return pathways 218, 212 in disposable portion 202 and non-disposable portion 204 which are fluidly connected by coupler 206. When the low pressure refrigerant is returned to the control console 102, the low pressure refrigerant is compressed such that the refrigerant can be pumped back to the cryoprobe 200 to supply further cooling at the conductive freeze tip. Return pathways 212, 218 can be coaxial with and surround the high pressure refrigerant delivery tube 210 and the capillary tube 220. The flow of high pressure and low pressure refrigerant can also be coaxial through coupler 206. The returning low pressure refrigerant will be at a lower temperature than the high pressure refrigerant and therefore will serve to further cool the high pressure refrigerant before it is expanded. This coaxial configuration also allows for the use of smaller cryoprobes than cryoprobes having the return and delivery tubes arranged in a side by side configuration throughout.

As illustrated in FIG. 2, both non-disposable portion 202 and disposable portion 204 can also include vacuum insulating spaces 208, 224 to insulate the refrigerant flowing through the cryoprobe 200. In some representative embodiments, vacuum insulating space 208 in non-disposable portion 202 can overlap vacuum insulating space 224 in disposable portion. Alternatively, the vacuum insulating spaces 208, 224 can abut against one another or against coupler 206. Vacuum insulating spaces 208, 224 help to prevent heat transfer between the returning, low pressure refrigerant and the ambient air or the body. By insulating the returning, low pressure refrigerant, the low pressure refrigerant is able to convect more heat from the high pressure refrigerant, which provides for greater cooling at the conductive freeze tip of cryoprobe 200.

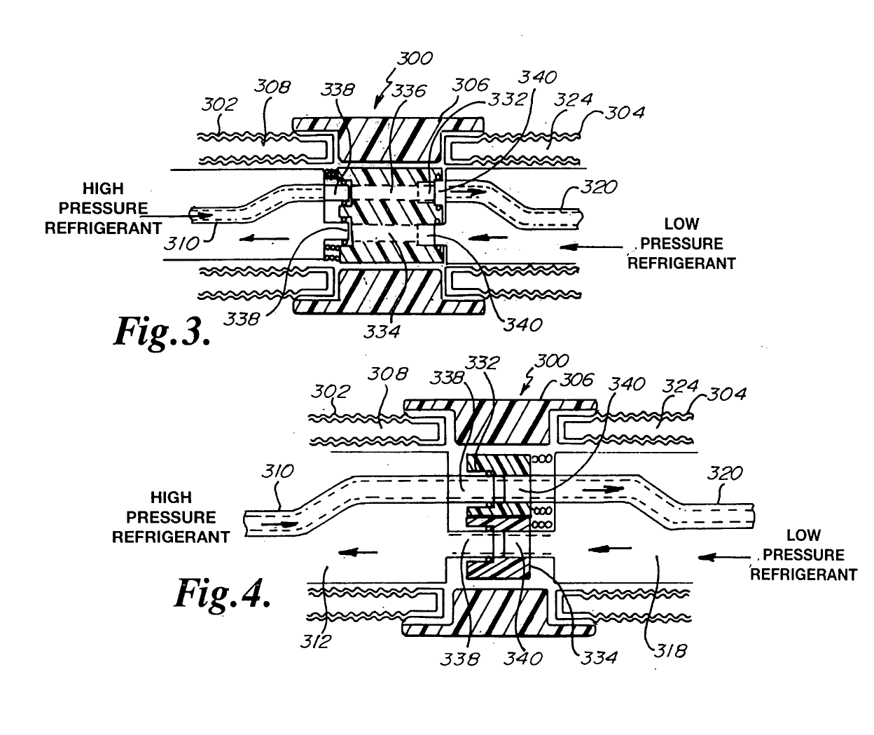

Referring now to FIGS. 3 and 4, there can be seen a portion of an embodiment of a cryoprobe 300 having side by side coupler ports 332, 334 for transferring the high pressure and low pressure refrigerant. High pressure refrigerant enters a delivery tube 310 through non-disposable portion 302 and flows through a capillary tube 320, or other Joule-Thompson expansion element, wherein the delivery tube 310 and capillary tube 320 are in fluid communication through coupler port 332. The high pressure refrigerant is expanded as it exits the capillary tube 320 and cools the conductive freeze tip of the cryoprobe 300. The resulting low pressure refrigerant then returns to the control console 102 through a return channel 318 in the disposable portion 304 where it flows into coupler port 334 and through a return channel 312 in non-disposable potion 302. Vacuum insulating spaces 308, 324 of non-disposable portion 302 and disposable portion 304 respectively, are depicted as abutting the coupler 306, but can alternatively overlap or abut one another.

Coupler 306 can interconnect the fluid channels of non-disposable portion 302 and disposable portion 304 in various ways. FIG. 3 depicts one representative embodiment having a dual male to female connection where male connections 338, 340 on both non-disposable portion 302 and disposable portion 304 are joined via a female union with coupler ports 332, 334. Within non-disposable portion 302, the two coaxial tubes 310, 312 diverge into two side by side tube connections 338 to mate with coupler ports 332, 334. Similarly, within disposable portion 304, the two coaxial tubes 318, 320 transition into two side by side tube connections 340 for joining with coupler ports 332, 334. The high pressure refrigerant flows through the delivery tube 310 and into a flow section 336 of coupler port 332 before entering capillary tube 320. Returning low pressure refrigerant flows through coupler port 334.

In another representative embodiment of cryoprobe 300, FIG. 4 depicts a male to female connection wherein male connections 340 of disposable portion 304 fluidly interconnect with female connections 338 of non-disposable portion 302. The high pressure refrigerant thus flows directly from delivery tube 310 into capillary tube 320. In other representative embodiments, male connections of non-disposable portion 302 can mate with female connections of disposable portions 304 and female ends on both non-disposable portion 302 and disposable portion 304 can be joined via a male union with coupler ports 332, 334. Finally, in some representative embodiments, both non-disposable portion 302 and disposable portion 304 can each include corresponding male and female connections.

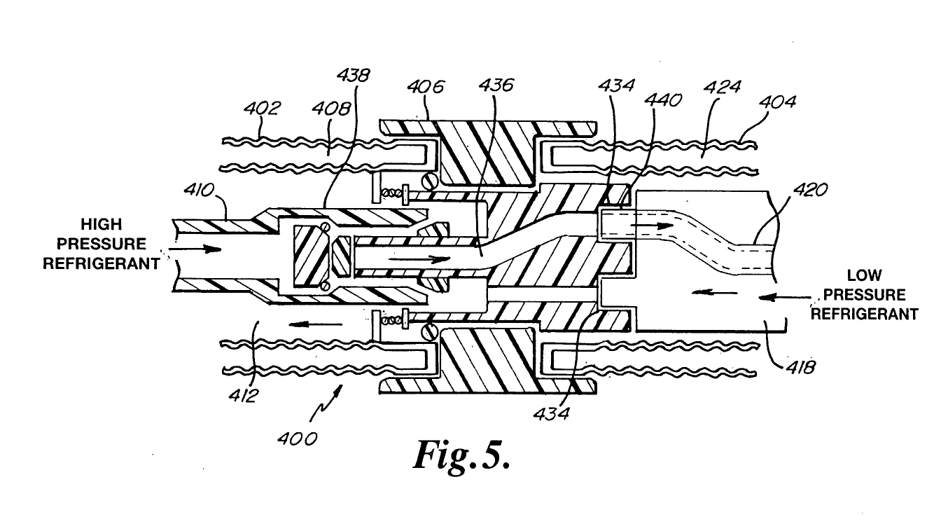

Referring now to FIG. 5, there is illustrated a portion of a cryoprobe 400 having a coaxial coupler attachment 438 with non-disposable portion 402 and a side by side coupler attachment 440 with disposable portion 404. Although coupler attachment 442 of disposable portion 404 is depicted as having a male to female connection with coupler ports 434, other representative embodiments can have a female to male connection. High pressure refrigerant can enter through a delivery tube 410 and travel through a coupler flow section 436 before entering a capillary tube 420. The returning refrigerant travels through a return channel 418 in disposable portion 404 and flows through coupler 406 into a return channel 412 in non-disposable portion 402 before returning to the system's control console. Although depicted as having a co-planar configuration, vacuum spaces 408, 424 can overlap one another.

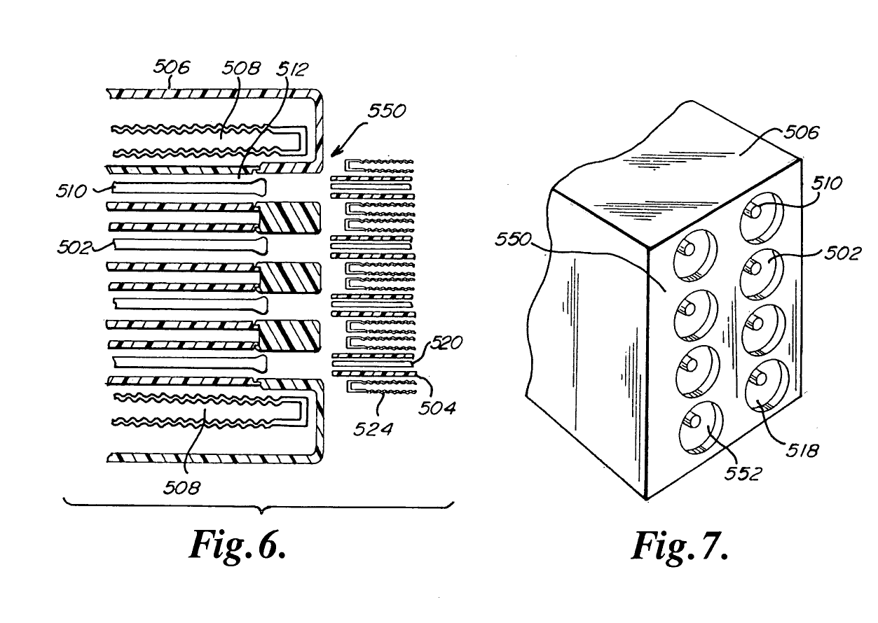

As described above, the disposable portion of each of the various cryoprobe embodiments can connect to the non-disposable portion through individual couplers. Alternatively, as shown in FIGS. 6-9, a plurality of disposable cryoprobe portions 504 can connect to a single manifold mounting plate 550 that can be held by the system's articulating arm 506. Manifold mounting plate 550 can be used to connect all non-disposable cryoprobe portions 502 in the system with disposable cryoprobe portions 504. In one presently preferred embodiment, non-disposable portions 502 are positioned in a 2×4 arrangement, as can be seen in FIG. 7 and FIG. 9.

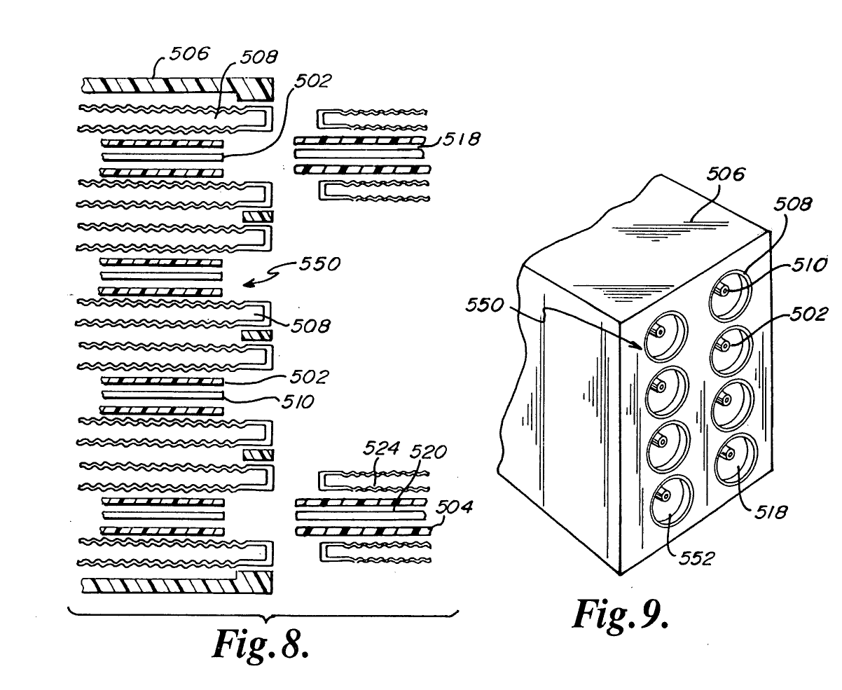

Each disposable portion 504 can plug into a manifold slot 552 of manifold mounting plate 550 to connect with a corresponding non-disposable portion 502. Refrigerant can then flow through delivery tubes 510 of non-disposable portions 502 into capillary tubes 520 of disposable portions 504 and through return channels 518 of disposable portions 504 into return channels 512 of non-disposable portions 502. In some representative embodiments, vacuum insulation 508 can be built into the manifold mounting plate 540, with a single line of insulation surrounding all of the non-disposable portions 502, as shown in FIG. 6. Alternatively, as shown in FIGS. 8 and 9, vacuum insulation 509 can surround each individual non-disposable portion 502. Vacuum insulation 509 can mate with the vacuum insulation 524 of each disposable portion 504.

Manifold mounting plate 540 can be utilized to centralize all cryoprobes and connections. This can be beneficial in preventing the otherwise independent lines from tangling with one another. Manifold mounting plate 540 also provides an interface that is reduced in size as compared to multiple cryoprobes each having an individual coupler.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

What is claimed is:

1. A manifold mounting plate for connecting a plurality of cryoprobes for use in a cryosurgical procedure, comprising:
a plurality of manifold slots; and
a non-disposable cryoprobe portion located in each manifold slot, each non-disposable cryoprobe portion including a delivery tube and a return channel.

2. The manifold mounting plate of claim 1; further comprising:
vacuum insulation for insulating the plurality of manifold slots.

3. The manifold mounting plate of claim 2, wherein the vacuum insulation individually surrounds each manifold slot.

4. The manifold mounting plate of claim 1, wherein the plurality of manifold slots are arranged in a 2×4 slot arrangement.

5. A cryosurgical system for performing cryosurgical procedures, comprising:
a control console including a refrigerant and a compressor for pressurizing the refrigerant;
a manifold mounting plate having a plurality of manifold slots, each manifold slot having a non-disposable cryoprobe portion including a delivery tube and a return channel fluidly connected to the control console; and
a plurality of disposable cryoprobe portions including a including an end refrigerant delivery tube and an end refrigerant return channel, wherein the plurality of disposable cryoprobe portions are fluidly interconnected to the non-disposable cryoprobe portions in each manifold slot.

6. The cryosurgical system of claim 5, wherein the manifold mounting plate further includes vacuum insulation for insulating the plurality of manifold slots.

7. The cryosurgical system of claim 6, wherein the vacuum insulation individually surrounds each manifold slot.

8. The cryosurgical system of claim 5, wherein the plurality of manifold slots are arranged in a 2 x 4 slot arrangement.

9. The cryosurgical system of claim 5, wherein the manifold mounting plate is operably mounted to an articulating arm.

10. The cryosurgical system of claim 5, wherein the end refrigerant delivery tube includes a capillary tube for expanding the refrigerant in the disposable cryoprobe portion.

11. A method of centralizing the connection of cryoprobes in a cryosurgical system comprising:
providing a manifold mounting plate having a plurality of manifold slots, each manifold slot including a non-disposable cryoprobe portion having a delivery tube and a return channel;
attaching at least one disposable cryoprobe portion to at least one of the non-disposable cryoprobe portion such that a capillary tube fluidly connects to the delivery tube and a return pathway fluidly connects to the return channel; and
supplying a high pressure refrigerant to the delivery tube such that the high pressure refrigerant is expanded in the capillary tube to cool a tip of the at least one disposable portion.

12. The method of claim 11, further comprising:
removing the at least one disposable cryoprobe portion from the manifold mounting plate; and
disposing of the at least one disposable cryoprobe portion.

13. The method of claim 11, further comprising:
mounting the manifold mounting plate to an articulating arm.

14. The method of claim 11, further comprising:
insulating the non-disposable cryoprobe portions.

15. The method of claim 14, wherein insulating the non-disposable cryoprobe portions includes individually insulating each non-disposable cryoprobe portion.
